SEQUENCE LISTING (3 sequences)

SEQ ID NO: 1 - protein, 1016 aa, Artificial Sequence
  SREBP2 protein
MDDSGELGGLETMETLTELGDELTLGDIDEMLQFVSNQVGEFPDLFSEQLCSSFPGSGGSGSSSGSSGSSSSSSNGRGSSSGAVDPSVQRSFTQVTLPSFSPSAASPQAPTLQVKVSPTSVPTTPRATPILQPRPQPQPQPQTQLQQQTVMITPTFSTTPQTRIIQQPLIYQNAATSFQVLQPQVQSLVTSSQVQPVTIQQQVQTVQAQRVLTQTANGTLQTLAPATVQTVAAPQVQQVPVLVQPQIIKTDSLVLTTLKTDGSPVMAAVQNPALTALTTPIQTAALQVPTLVGSSGTILTTMPVMMGQEKVPIKQVPGGVKQLEPPKEGERRTTHNIIEKRYRSSINDKIIELKDLVMGTDAKMHKSGVLRKAIDYIKYLQQVNHKLRQENMVLKLANQKNKLLKGIDLGSLVDNEVDLKIEDFNQNVLLMSPPASDSGSQAGFSPYSIDSEPGSPLLDDAKGDFAAAAGNLQTCLAVLGRALPTSRLDLACSLSWNVIRYSLQKLRLVRWLLKKVFQCRRATPATEAGFEDEAKTSARDAALAYHRLHQLHITGKLPAGSACSDVHMALCAVNLAECAEEKIPPSTLVEIHLTAAMGLKTRCGGKLGFLASYFLSRAQSLCGPEHSAVPDSLRWLCHPLGQKFFMERSWSVKSAAKESLYCAQRNPADPIAQVHQAFCKNLLERAIESLVKPQAKKKAGDQEEESCEFSSALEYLKLLHSFVDSVGVMSPPLSRSSVLKSALGPDIICRWWTSAITVAISWLQGDDAAVRSHFTKVERIPKALEVTESPLVKAIFHACRAMHASLPGKADGQQSSFCHCERASGHLWSSLNVSGATSDPALNHVVQLLTCDLLLSLRTALWQKQASASQAVGETYHASGAELAGFQRDLGSLRRLAHSFRPAYRKVFLHEATVRLMAGASPTRTHQLLEHSLRRRTTQSTKHGEVDAWPGQRERATAILLACRHLPLSFLSSPGQRAVLLAEAARTLEKVGDRRSCNDCQQMIVKLGGGTAIAAS

SEQ ID NO: 2 - DNA, 3051 bp, Artificial Sequence
  SREBP2 mRNA
atggacgacagcggcgagctgggtggtctggagaccatggagaccctcacggagctgggcgacgagctgaccctgggagacatcgacgagatgctgcaatttgtcagtaatcaagtgggagagttccctgacttgttttcagaacagctgtgtagctcctttcctggcagtggtggtagtggtagcagcagcggcagcagtggcagcagcagcagcagcagcaatggcaggggcagcagcagcggagctgtggacccttcagtgcaacggtcattcacccaggtcacattaccttccttctctccctcggcggcctccccacaggctccaactctgcaagtcaaggtttctcccacctcagttcccaccacacccagggcaactcctattcttcagccccgcccccagccccagcctcaacctcaaactcagctgcaacaacagacggtaatgatcacgccaacattcagcaccactccgcagacgaggatcatccagcagcctttgatataccagaatgcagctactagctttcaagtccttcagcctcaagtccaaagcctggtgacatcctcccaggtacagccggtcaccattcagcagcaggtgcagacagtacaggcccagcgggtgctgacacaaacggccaatggcacgctgcagacccttgccccggctacggtgcagacagttgctgcgccacaggtgcagcaggtcccggtcctggtccagcctcagatcatcaagacagattcccttgttttgaccacactgaagacagatggcagccctgttatggctgcggtccagaacccggccctcaccgccctcaccacccctatccagacggctgcccttcaagtaccaaccctggtgggcagcagtgggaccattctgaccacaatgcctgtaatgatggggcaagagaaagtgcccattaagcaggtacctgggggagtcaagcagcttgagccccccaaagaaggagaaaggcggacaacccataatatcattgagaaacgatatcgctcctccatcaatgacaaaatcatcgaattgaaagacctggtcatggggacagacgccaagatgcacaagtctggcgttctgaggaaggccattgattacatcaaatacttgcagcaggtcaatcataaactgcgccaggagaacatggtgctgaagctggcaaatcaaaagaacaagcttctaaagggcatcgacctaggcagtctggtggacaatgaggtggacctgaagatcgaggactttaatcagaatgtccttctgatgtcccccccagcctctgactcagggtcccaggctggcttctctccctactccattgactctgagccaggaagccctctattggatgatgcaaagggagattttgcagctgctgccggcaacctacaaacctgcctggcagttttgggccgggcactgcccacctcccgcctggacctggcctgcagcctctcctggaacgtgatccgctacagcctgcagaagctacgcctggtgcgctggctgctcaagaaagtcttccagtgccggcgggccacgccagccactgaggcaggctttgaagacgaagctaagaccagcgcccgggatgcggctctggcctatcaccggctgcaccagctgcacatcacagggaagcttcctgcaggatccgcctgttccgatgtacacatggcgttgtgtgccgtgaacctggctgaatgtgcagaggagaagatcccaccgagcacactggttgagatccatctgactgctgccatggggctcaagacccggtgtggaggcaagctgggcttcctggccagctacttcctcagccgagcccagagcctgtgtggccccgagcacagtgctgttcctgactccctgcgctggctctgccaccccctgggccagaagtttttcatggagcggagctggtctgtgaagtcagctgccaaggagagtctatactgtgcccagaggaacccagctgaccccattgcgcaggtccaccaggccttctgcaagaacctgctggagcgagctatagagtccttggtgaaacctcaggccaagaagaaggctggagaccaggaagaagagagctgtgaattctccagtgctctggagtacttgaaattacttcattcttttgtggactctgtgggggttatgagccccccactctccaggagctccgtgctcaagtccgccctgggtccagacatcatctgtcggtggtggacgtctgcaatcactgtggccatcagctggctccagggagacgatgcagctgtgcgctctcattttaccaaagtggaacgcatccccaaggccctggaagtgacagagagccccctggtgaaggccatcttccatgcctgcagagccatgcatgcctcactccctgggaaagcagatgggcagcagagttccttctgccattgcgagagggccagtggccacctatggagcagcctcaacgtcagtggggccacctctgaccctgccctcaaccacgtggtccagctgctcacctgtgacctgctactgtcgctacggacagcgctctggcaaaaacaggccagtgccagccaggctgtgggggagacctaccacgcgtcaggcgctgaactggcgggcttccaacgggacctgggcagcctgcgcaggctggcacacagcttccgcccagcataccgcaaggtgttcctgcatgaagccaccgtgcgcctgatggcaggagccagccccacccgcacccaccagctgctggaacacagcctgcggcggcgcaccacgcagagcaccaagcacggagaggtggatgcctggcccggccagcgagagcgggccaccgccatcctgctggcctgccgccacctgcccctctccttcctctcctccccgggccagcgggcagtgctgctggccgaagctgcccgcaccctggagaaggtgggcgaccggcgctcctgcaacgactgccagcagatgattgttaagctgggtggtggcactgccattgccgcctcctga

SEQ ID NO: 3 - protein, 378 aa, Artificial Sequence
  SREBP2 C-terminal amino acid sequence
VFQCRRATPATEAGFEDEAKTSARDAALAYHRLHQLHITGKLPAGSACSDVHMALCAVNLAECAEEKIPPSTLVEIHLTAAMGLKTRCGGKLGFLASYFLSRAQSLCGPEHSAVPDSLRWLCHPLGQKFFMERSWSVKSAAKESLYCAQRNPADPIAQVHQAFCKNLLERAIESLVKPQAKKKAGDQEEESCEFSSALEYLKLLHSFVDSVGVMSPPLSRSSVLKSALGPDIICRWWTSAITVAISWLQGDDAAVRSHFTKVERIPKALEVTESPLVKAIFHACRAMHASLPGKADGQQSSFCHCERASGHLWSSLNVSGATSDPALNHVVQLLTCDLLLSLRTALWQKQASASQAVGETYHASGAELAGFQRDLGSL

(19) United States
(12) Patent Application Publication
(10) Pub. No.: US 2023/0333109 A1
(43) Pub. Date: Oct. 19, 2023

(54) COMPOSITION FOR DIAGNOSING INFECTIOUS DISEASES COMPRISING AGENT FOR MEASURING EXPRESSION LEVEL OF SREBP2

(71) Applicants: KOREA RESEARCH INSTITUTE OF BIOSCIENCE AND BIOTECHNOLOGY, Yuseong-gu, Daejeon (KR); KYUNGPOOK NATIONAL UNIVERSITY INDUSTRY-ACADEMIC COOPERATION FOUNDATION, Buk-gu, Daegu (KR)

(72) Inventors: Young Kyo SEO, Daejeon (KR); Jong Sup BAE, Buk-gu (KR); Wonhwa LEE, Yuseong-gu (KR); Youngbum YOO, Yuseong-gu (KR)

(73) Assignees: KOREA RESEARCH INSTITUTE OF BIOSCIENCE AND BIOTECHNOLOGY, Yuseong-gu, Daejeon (KR); KYUNGPOOK NATIONAL UNIVERSITY INDUSTRY-ACADEMIC COOPERATION FOUNDATION, Buk-gu, Daegu (KR)

(21) Appl. No.: 18/022,050

(22) PCT Filed: Aug. 20, 2021
(86) PCT No.: PCT/KR2021/011114

§ 371 Date: Feb. 17, 2023

(30) Foreign Application Priority Data
Aug. 20, 2020 (KR) 10-2020-0104962

(51) Int. Cl. G01N 33/569

(52) U.S. Cl. CPC G01N 33/56983; G01N 2333/165

(57) ABSTRACT

The present invention relates to a composition for diagnosing infectious diseases, comprising an agent for measuring the expression level of sterol regulatory element-binding protein 2 (SREBP2) and, more specifically, to a composition for diagnosing infectious diseases or diagnosing the severity thereof, the composition comprising an agent for measuring the expression level of sterol regulatory element-binding protein 2 (SREBP2) or a C-terminal peptide thereof.

### TECHNICAL FIELD

This application claims the priority of Korean Patent Application No. 10-2020-0104962, filed on Aug. 20, 2020, the entirety of which is a reference of the present application.

The present invention relates to a composition for diagnosing infectious diseases comprising an agent for measuring the expression level of sterol regulatory element binding protein 2 (SREBP2), and more particularly, to a composition for diagnosing infectious diseases or diagnosing the severity thereof comprising an agent for measuring the expression level of sterol regulatory element binding protein 2 (SREBP2) or a C-terminal peptide thereof.

### BACKGROUND ART

Infectious diseases are diseases that develop when foreign substances such as germs, bacteria, and viruses appear and start to live in blood, body fluids, and tissues, and diseases that can lose life when not accurately identified and appropriately treated. It seems that the prevalence of infectious diseases generally decreases with the improvement of sanitation standard, but the threat of infectious diseases with fatal consequences is increasing due to misuse of antibiotics, increased use of immunosuppressants by transplantation, decreased immunity due to chemotherapy, and increased number of patients with underlying diseases such as diabetes and hypertension.

In particular, most infectious diseases accompany inflammatory responses at infection sites, and some of the infectious diseases cause a systemic inflammatory response, leading to fatal results. In addition, since infectious patients due to infection may die, it is important to start appropriate antibiotic treatment as soon as possible, so that accurate diagnosis and severity prediction are essential for the survival of infectious patients.

Meanwhile, the pandemic of a new human coronavirus has recently spread rapidly around the world to pose a threat to global health. Surprisingly, the symptoms observed in patients infected with SARS-CoV-2 are similar to other viral infections such as severe acute respiratory syndrome (SARS) and Middle East respiratory syndrome (MERS) reported previously. Pneumonia-related symptoms such as acute respiratory distress syndrome (ARDS), cytokine release syndrome (CRS), multi-organ failure (MOF), and sepsis were observed in many of these patients. No approved therapies or effective therapeutic agents have been identified for the treatment of SARS-CoV-2 infection. In response to the epidemic of SARS-CoV-2 infection, the global effort for vaccine research and development is unprecedented in speed and scale. However, since the vaccine development still requires a lot of time, there is an urgent need to develop effective therapies to reduce the mortality rate of patients with SARS-CoV-2 infection.

On the other hand, sterol regulatory element binding proteins (SREBPs) are well known as basic-helix-loop helix-leucine zipper transcription factors that regulate the expression of genes involved in lipid cholesterol biosynthesis. These SREBP transcription factors have been reported to regulate the expression of lipid cholesterol and fatty acid genes through an MAPK activation pathway. The SREBP transcription factor is an important regulator of lipid biosynthesis and sterol homeostasis in eukaryotes, and in mammals, SREBP is highly active in a feeding state to promote the expression of cholesterogenic and adipogenic genes involved in the fat state. However, recently, it has been reported that various pathogenic processes such as endoplasmic reticulum (ER) stress, inflammation, apoptosis, and autophagy are related to SREBP, and the expression of SREBP is also involved in the hardening process of damaged tissue.

According to a recently published study, it was confirmed that the transcription of Sestrin1 (SESN1) in cells was regulated by SREBP2, SESN1 was a target gene of SREBP2, and SESN1 inhibited cholesterol biosynthesis. SREBP2 inhibits lipid biosynthesis by increasing the expression levels of SESN1 and proprotein convertase subtilisin kexin type 9 (PCSK9). To observe whether SESN1 affects hepatic cholesterol biosynthesis, mice were treated with Lovastatin to inhibit HMGCR activity and block cholesterol biosynthesis. According to recently published data, phosphorylated AMPK significantly decreased in the liver of SESN1−/− mice after cholesterol feeding, and mTORC1 did not change, indicating that SESN1 functions to inhibit cholesterol biosynthesis through the activation of an AMP kinase pathway.

However, it is still unknown how SREBP2 is involved in the induction of cell and tissue damage and ultimately inflammation during the infection process.

### DISCLOSURE

### Technical Problem

Therefore, the present inventors have found that during a study on a correlation between infectious diseases and SREBP2, in biological samples from patients with infectious diseases, the expression level of SREBP2 or the expression level of a C-terminal peptide of SREBP2 was significantly higher than that of normal subjects, and the expression level thereof was very closely related with the severity of the diseases.

Accordingly, an object of the present invention is to provide a composition for diagnosing an infectious disease comprising an agent for measuring the expression level of sterol regulatory element-binding protein 2 (SREBP2) or mRNA.

In addition, an object of the present invention is to provide a composition for diagnosing an infectious disease consisting of an agent for measuring the expression level of sterol regulatory element-binding protein 2 (SREBP2) or mRNA.

In addition, an object of the present invention is to provide a composition for diagnosing an infectious disease essentially consisting of an agent for measuring the expression level of sterol regulatory element-binding protein 2 (SREBP2) or mRNA.

Another object of the present invention is to provide a kit for diagnosing an infectious disease comprising an agent for measuring the expression level of sterol regulatory element-binding protein 2 (SREBP2) or mRNA.

Yet another object of the present invention is to provide a method for detecting SREBP2 to provide information necessary for the diagnosis of an infectious disease, comprising (a) measuring the expression level of sterol regulatory element binding protein 2 (SREBP2) or mRNA in a biological sample provided from a patient suspected of having an infectious disease; and (b) comparing the expression level of the SREBP2 protein or mRNA with that of a normal subject and determining an infectious disease when the expression level of the SREBP2 protein or mRNA is increased compared to the normal subject.

Yet another object of the present invention is to provide a use of an agent for measuring the expression level of sterol regulatory element-binding protein 2 (SREBP2) or mRNA to prepare an agent for diagnosing an infectious disease.

Yet another object of the present invention is to provide a method for diagnosing an infectious disease comprising the steps of:
(a) measuring the expression level of sterol regulatory element binding protein 2 (SREBP2) or mRNA in a biological sample obtained from a subject suspected of having an infectious disease;
(b) measuring the expression level of the SREBP2 protein or mRNA in step (a);
(c) comparing the expression level of the SREBP2 protein or mRNA with that of a normal subject; and
(d) diagnosing an infectious disease when the expression level of the SREBP2 protein or mRNA is increased compared to the normal subject in step (c).

### Technical Solution

In order to achieve the object of the present invention, an aspect of the present invention provides a composition for diagnosing an infectious disease comprising an agent for measuring the expression level of sterol regulatory element-binding protein 2 (SREBP2) or mRNA.

In addition, the present invention provides a composition for diagnosing an infectious disease consisting of an agent for measuring the expression level of sterol regulatory element-binding protein 2 (SREBP2) or mRNA.

In addition, the present invention provides a composition for diagnosing an infectious disease essentially consisting of an agent for measuring the expression level of sterol regulatory element-binding protein 2 (SREBP2) or mRNA.

In order to achieve another object of the present invention, the present invention provides a kit for diagnosing an infectious disease comprising an agent for measuring the expression level of sterol regulatory element-binding protein 2 (SREBP2) or mRNA.

In order to achieve yet another object of the present invention, the present invention provides a method for detecting SREBP2 to provide information necessary for the diagnosis of an infectious disease, comprising (a) measuring the expression level of sterol regulatory element binding protein 2 (SREBP2) or mRNA in a biological sample provided from a patient suspected of having an infectious disease; and (b) comparing the expression level of the SREBP2 protein or mRNA with that of a normal subject and determining an infectious disease when the expression level of the SREBP2 protein or mRNA is increased compared to the normal subject.

In order to achieve yet another object of the present invention, the present invention provides a use of an agent for measuring the expression level of sterol regulatory element-binding protein 2 (SREBP2) or mRNA to prepare an agent for diagnosing an infectious disease.

In order to achieve yet another object of the present invention, the present invention provides a method for diagnosing an infectious disease comprising steps of:
(a) measuring the expression level of sterol regulatory element binding protein 2 (SREBP2) or mRNA in a biological sample obtained from a subject suspected of having an infectious disease;
(b) measuring the expression level of the SREBP2 protein or mRNA in step (a);
(c) comparing the expression level of the SREBP2 protein or mRNA with that of a normal subject; and
(d) diagnosing an infectious disease when the expression level of the SREBP2 protein or mRNA is increased compared to the normal subject in step (c).

Hereinafter, the present invention will be described in detail.

According to one embodiment of the present invention, it was confirmed that the expression levels of SREBP2 protein and mRNA in the blood of patients with infectious diseases were significantly increased compared to normal subjects, and the increased level of SREBP2 showed a tendency to be proportional to the severity of the diseases. In addition, the C-terminal peptide of the SREBP2 protein showed the same tendency, but since SREBP2 has the characteristic of being released from cells, it was confirmed that infectious diseases and the severity thereof may be diagnosed very accurately with a simple blood test.

Accordingly, the present invention provides a composition for diagnosing an infectious disease comprising an agent for measuring the expression level of sterol regulatory element-binding protein 2 (SREBP2) or mRNA.

In the present invention, the SREBP2 is a transcription factor that regulates lipid homeostasis and metabolism, and precisely regulates the expression of enzymes required for the synthesis of endogenous cholesterol, fatty acids, triacylglycerols, and phospholipids. The SREBP2 is also known as sterol regulatory element binding transcription factor 2 (SREBF2) and encoded by an SREBP2 gene in humans. An amino acid sequence and an mRNA base sequence of the SREBP2 protein are known as Genebank accession numbers NP_004590.2 (protein), NM_004599.4 (mRNA), etc., and preferably represented by SEQ ID NO: 1 (protein) or SEQ ID NO: 2 (mRNA).
SEQ ID NO: 1; Protein of full-length SREBP2
SEQ ID NO: 2; full-length SREBF-2 mRNA sequences

In the present invention, the ‘expression’ means that a protein or nucleic acid is produced in cells. The ‘protein’ is used interchangeably with a ‘polypeptide’ or ‘peptide’, and for example, refers to a polymer of amino acid residues as commonly found in the protein in a natural state. The ‘polynucleotide’ or ‘nucleic acid’ refers to deoxyribonucleotide (DNA) or ribonucleotide (RNA) in a single- or double-stranded form. Unless otherwise limited, the ‘polynucleotide’ also includes known analogs of natural nucleotides that hybridize with nucleic acids in a manner similar to naturally produced nucleotides. The ‘mRNA’ is RNA that transmits genetic information (gene-specific base sequence) to ribosomes that specify amino acid sequences from a specific gene during protein synthesis.

The ‘diagnosis’ means to confirm the presence or characteristics of a pathological condition. The diagnosis in the present invention is to determine the pathology presence or development of infectious diseases by measuring the expression level of SREBP2, that is, the level of the SREBP2 protein or mRNA.

In one aspect of the present invention, the agent for measuring the expression level of the SREBP2 protein may be an antibody, a fragment of the antibody or an aptamer that specifically binds to the SREBP2 protein.

The ‘antibody’ refers to immunoglobulin that specifically binds to an antigenic site. The antibody of the present invention is an antibody that specifically binds only to the SREBP2 protein without reacting with other proteins including other types of SREBPs other than SREBP2. The SREBP2 antibody may be prepared by cloning the SREBP2 gene into an expression vector to obtain a protein encoded by the gene, and using a conventional method in the art from the obtained protein. An SREBP2 protein-specific antibody may also be prepared using a fragment of the SREBP2 protein including an SREBP2 antigenic site.

The form of the antibody of the present invention is not particularly limited, and includes a polyclonal antibody or a monoclonal antibody. In addition, as long as the antibody has antigen-antibody binding properties, a part of the overall antibody is also included in the antibody of the present invention, and all types of immunoglobulin antibodies that specifically bind to SREBP2 are included. For example, the antibody of the present invention includes not only an intact antibody with two full-length light chains and two full-length heavy chains, but also functional fragments of an antibody molecule, that is, Fab, F(ab′), F(ab′)2 and Fv with an antigen-binding function. Furthermore, the antibody of the present invention includes special antibodies such as humanized antibodies and chimeric antibodies and recombinant antibodies, as long as the antibody may specifically bind to the SREBP2 protein.

In the present invention, the SREBP2 protein preferably comprises a human SREBP2 amino acid sequence represented by SEQ ID NO: 1, and in the present invention, the antibody that specifically binds to the SREBP2 protein may be preferably an antibody that specifically binds to a protein having the amino acid sequence represented by SEQ ID NO: 1. The diagnostic composition of the present invention comprising the SREBP2-specific antibody as the agent for measuring the expression level of SREBP2 may further include an agent required for a known method for detecting a protein, and the level of the SREBP2 protein may be measured using the known method for detecting the protein using the composition without limitation.

In the present invention, the ‘aptamer’ refers to single-stranded DNA (ssDNA) or RNA having high specificity and affinity for a specific substance. The aptamer is very high in the affinity for the specific substance and stable, and may be synthesized by a relatively simple method, and since various modifications are possible to increase the binding force, and cells, proteins, and even small organic substances may be target substances, the specificity and stability thereof are very high compared to antibodies that have already been developed. In the present invention, the type and form of the aptamer are not particularly limited as long as the aptamer may bind to SREBP2.

In another aspect of the present invention, the agent for measuring the expression level of the SREBP2 mRNA may be a primer pair, a probe, or a combination thereof that specifically binds to the SREBP2 mRNA.

The SREBP2 mRNA may be derived from mammals including humans, and preferably may include a human SREBP2 mRNA base sequence represented by SEQ ID NO: 2. The diagnostic composition of the present invention comprising the SREBP2 mRNA-specific probe or primer set as the agent for measuring the expression level of SREBP2 may further include an agent required for a known method for detecting RNA. The level of the SREBP2 mRNA in a subject may be measured using the known method for detecting RNA without limitation using the present composition.

The ‘primer’ is a short single stranded oligonucleotide that acts as a starting point for DNA synthesis. The primer specifically binds to a polynucleotide as a template in suitable buffer and temperature conditions, and DNA polymerase is linked to the primer by adding a nucleoside triphosphate having a complementary base to the template DNA to synthesize DNA. The primer generally consists of 15 to 30 base sequences, and a melting temperature (Tₘ) binding to a template strand varies depending on the base composition and length.

The sequence of the primer is not required to have a sequence that is completely complementary to some base sequences of the template, and it is sufficient to have sufficient complementarity within a range capable of hybridizing with the template to function unique to the primer. Therefore, the primer for measuring the expression level of the SREBP2 mRNA in the present invention has no need to have a sequence perfectly complementary to an SREBP2 gene sequence, and is enough to have the length and complementarity suitable for the purpose of measuring the amount of SREBP2 mRNA by amplifying a specific section of SREBP2 mRNA or SREBP2 cDNA through DNA synthesis. The primer for the amplification reaction consists of a set (pair) that complementarily binds to a template (or sense) and an opposite side (antisense) of both ends of the specific section of SREBP2 mRNA to be amplified, respectively. The primer may be easily designed by those skilled in the art by referring to an SREBP2 mRNA or cDNA base sequence.

In the present invention, the primer may preferably be one set or one pair that binds specifically to an SREBP2 mRNA base sequence represented by SEQ ID NO: 2.

The ‘probe’ refers to a fragment of a polynucleotide such as RNA or DNA of several to hundreds of base pairs in length capable of specifically binding to mRNA, complementary DNA (cDNA), or the like of a specific gene and is labeled to confirm the presence or absence, an expression level, etc. of mRNA or cDNA to be bound. For the purpose of the present invention, a probe complementary to SREBP2 mRNA may be used for diagnosis of an infectious disease by performing a hybridization reaction with a sample of a subject to measure the expression level of SREBP2 mRNA. The selection and hybridization conditions of the probe may be appropriately selected according to techniques known in the art.

The primer or probe of the present invention may be chemically synthesized using a phosphoramidite solid support synthesis method or other well-known methods. In addition, the primer or probe may be variously modified according to methods known in the art within a range that does not interfere with hybridization with SREBP2 mRNA. Examples of such modifications include methylation, encapsulation, substitution of one or more homologs of natural nucleotides, and modifications between nucleotides, such as uncharged linkers (e.g., methyl phosphonate, phosphotriester, phosphoroamidate, carbamate, etc.) or charged linkers (e.g., phosphorothioate, phosphorodithioate, etc.), binding of a labeling material using fluorescence or enzymes, and the like.

According to one embodiment of the present invention, it was confirmed that the C-terminal peptide of SREBP2 was expressed significantly high in the blood and PBMC of infected patients. In particular, since the C-terminal peptide of SREBP2 can be detected in the blood of the patients to diagnose an infectious disease, it can be very useful in that rapid and non-invasive diagnosis is possible.

In the present invention, the SREBP2 C-terminal peptide refers to a peptide including amino acids at positions 639 to 1031 in the full-length sequence of the SREBP2 protein of SEQ ID NO: 1, preferably a peptide consisting of an amino acid sequence of SEQ ID NO: 3.

SEQ ID NO: 3 SREBP2 C-terminal of 639-1031aa region

In the present invention, the ‘infection’ means that one or two or more types of exogenous bacteria (including bacteria, gram-negative bacteria, or gram-positive bacteria), viruses, and fungi invade the body to settle, proliferate, and become parasitic, and the infectious diseases may be all diseases caused by responses in vivo as a result of infection with pathogens. The responses resulting from the infectious diseases may include inflammation, pain, fever, fatigue, edema, and drops in blood pressure.

In the present invention, the infectious disease may be preferably an infectious inflammatory disease. The infectious disease may be more preferably at least one selected from the group consisting of sepsis, septic shock, severe acute respiratory syndrome coronavirus 2 (SARS-CoV-2) infection, severe acute respiratory syndrome coronavirus (SARS-CoV) infection, Middle East respiratory syndrome (MERS), salmonellosis, food poisoning, typhoid, paratyphoid, systemic inflammatory response syndrome (SIRS), multiple organ dysfunction syndrome (MODS), pneumonia, pulmonary tuberculosis, tuberculosis, cold, influenza, airway infection, rhinitis, nasopharyngitis, otitis media, bronchitis, lymphadenitis, parotitis, lymphadenitis, cheilitis, stomatitis, arthritis, myositis, dermatitis, vasculitis, gingivitis, periodontitis, keratitis, conjunctivitis, wound infection, peritonitis, hepatitis, osteomyelitis, cellulitis, meningitis, encephalitis, brain abscess, encephalomyelitis, meningitis, osteomyelitis, nephritis, carditis, endocarditis, enteritis, gastritis, esophagitis, duodenitis, colitis, urinary tractitis, cystitis, vaginitis, cervicitis, salpingitis, erythema infectious, dysentery, abscesses and ulcers, bacteremia, diarrhea, dysentery, enterogastritis, gastroenteritis, genitourinary abscess, open wound or injury infection, suppurative inflammation, abscess, boil, pyoderma, impetigo, folliculitis, cellulitis, postoperative wound infection, skin laceration syndrome, skin burn syndrome, thrombotic thrombocytopenia, hemolytic uremic syndrome, renal failure, pyelonephritis, glomerulonephritis, nervous system abscess, otitis media, sinusitis, pharyngitis, tonsillitis, mastoiditis, cellulitis, devotion infection, dacryocystitis, pleurisy, abdominal abscess, liver abscess, cholecystitis, spleen abscess, pericarditis, myocarditis, placenta, amniotic fluid infection, mammitis, mastitis, puerperal fever, toxic shock syndrome, lyme disease, gas gangrene, atherosclerosis, Mycobacterium avium syndrome (MAC), enterohemorrhagic Escherichia coli (EHEC) infection, enteropathogenic Escherichia coli (EPEC) infection, enterohemorrhagic Escherichia coli infection (EIEC), methicillin-resistant Staphylococcus aureus (MRSA) infection, vancomycin-resistant Staphylococcus aureus (VRSA) infection, and listerosis. The infectious disease may be most preferably sepsis, septic shock, and severe acute respiratory syndrome coronavirus 2 (SARS-CoV-2) infection, but is not limited thereto.

In the present invention, the ‘sepsis’ is a systemic inflammatory response syndrome that appears as a complication of an infectious disease, and if the cause thereof is not diagnosed early, quickly and accurately, the sepsis is a fatal disease that is developed into severe sepsis or septic shock, multiple organ dysfunction syndrome (MODS) caused to functional disorders such as lungs, kidneys, liver, and circulatory system, disseminated intravascular coagulation syndrome (DIC), acute respiratory distress syndrome (ARDS) or acute kidney injury (AKI), leading to death.

In the present invention, the sepsis includes sepsis related to the final stage of sepsis, severe sepsis, septic shock and multiple organ dysfunction syndrome (MODS) accompanying sepsis, disseminated intravascular coagulation syndrome (DIC), acute respiratory distress syndrome (ARDS) or acute kidney injury (AKI), but is not limited thereto and includes all stages of the sepsis.

In the present invention, the severe acute respiratory syndrome virus 2 (SARS-CoV-2) is an RNA virus belonging to Corona, and called SARS-CoV-2, COVID-19, Covid-19, Corona-19, 2019-nCoV, 2019 new coronavirus, coronavirus disease 2019, etc. In the present invention, the SARS-CoV-2 may cause various mutations during the infection process to generate variants, and these variants may also be included in the SARS-CoV-2 of the present invention.

According to one embodiment of the present invention, it was confirmed that the expression level of SREBP2 or the C-terminal peptide thereof was significantly increased in a biological sample provided from an infectious patient as the disease severity of the infectious patient increased.

Accordingly, the diagnostic composition provided by the present invention may be characterized to predict the severity of an infectious disease as well as to diagnose the infectious diseases.

In addition, the present invention provides a kit for diagnosing an infectious disease comprising an agent for measuring the expression level of sterol regulatory element-binding protein 2 (SREBP2) or mRNA.

The diagnostic kit of the present invention may include not only an antibody, a fragment of the antibody, and an aptamer recognizing selectively the SREBP2 protein or a primer and a probe recognizing SREBP2 mRNA as a marker, but also a composition, a solution or a device of one type one or more other ingredients suitable for an analytical method, in order to measure the expression level of SREBP2.

As a specific embodiment, the diagnostic kit may be a diagnostic kit characterized to include essential elements required for performing a reverse transcription polymerase reaction. The reverse transcription polymerase reaction kit includes each primer pair specific for a marker gene. The primer is a nucleotide having a sequence specific for the nucleic acid sequence of each marker gene, and is about 7 bp to 50 bp in length, more preferably about 10 bp to 30 bp in length. In addition, a primer specific for a nucleic acid sequence of a control gene may be included. Other reverse transcription polymerase reaction kits may include test tubes or other suitable containers, a reaction buffer (the pH and magnesium concentration are various), deoxynucleotides (dNTPs), enzymes such as Taq-polymerase and reverse transcriptase, DNAse, an RNAse inhibitor DEPC-water, sterilized water, etc.

Yet another aspect may be a diagnostic kit comprising essential elements required to perform a DNA chip. The DNA chip kit may include a substrate to which cDNA or oligonucleotides corresponding to genes or fragments thereof are attached, and a reagent, an agent, an enzyme, and the like for preparing a fluorescently labeled probe. In addition, the substrate may include a cDNA or oligonucleotide corresponding to the control gene or a fragment thereof.

Most preferably, the diagnostic kit may be a diagnostic kit comprising essential elements required to perform ELISA. The ELISA kit includes an antibody specific for a marker protein. The antibody is an antibody having high specificity and affinity for each marker protein and little cross-reactivity to other proteins, and includes monoclonal antibodies, polyclonal antibodies, or recombinant antibodies. In addition, the ELISA kit may include an antibody specific for a control protein. In addition, the ELISA kit may include reagents capable of detecting bound antibodies, for example, labeled secondary antibodies, chromophores, enzymes (in the form conjugated with antibodies), substrates thereof or other materials capable of binding to the antibodies, and the like. In addition, the kit of the present invention may include a washing solution or an eluent capable of removing substrates to color-react with the enzymes, non-binding proteins, and the like and retaining only the bound protein markers.

The present invention also provides a method for detecting SREBP2 to provide information necessary for the diagnosis of an infectious disease, comprising (a) measuring the expression level of sterol regulatory element binding protein 2 (SREBP2) or mRNA in a biological sample provided from a patient suspected of having an infectious disease; and (b) comparing the expression level of the SREBP2 protein or mRNA with that of a normal subject and determining an infectious disease when the expression level of the SREBP2 protein or mRNA is increased compared to the normal subject.

The present inventors first found that SREBP2 may function as a novel infectious disease marker, and provides a method for providing information necessary for the diagnosis of an infectious disease by measuring the expression level of SREBP2. Hereinafter, the method of the present invention will be described step by step.

(a) measuring the expression level of sterol regulatory element binding protein 2 (SREBP2) or mRNA in a biological sample provided from a patient suspected of having an infectious disease;

The biological sample may be used without limitation as long as the biological sample is collected from a subject to be diagnosed with an infectious disease, and for example, may include cells or tissues obtained by biopsy, etc., blood, plasma, serum, saliva, nasal fluid, sputum, synovial fluid, amniotic fluid, ascites, cervical or vaginal secretions, urine and cerebrospinal fluid, etc. Preferably, the biological sample may be blood, plasma, or serum.

The level of the SREBP2 protein may be detected or measured using an antibody that specifically binds to the SREBP2 protein. The SREBP2 protein-specific antibody is as described in the diagnostic composition of the present invention. As the method for measuring the expression level of the SREBP2 protein, methods known in the art may be used without limitation, and the method includes, for example, western blotting, dot blotting, enzyme-linked immunosorbent assay (ELISA), radioimmunoassay (RIA), radioimmunoassay, octeroney immunodiffusion method, rocket immunoelectrolysis, immunohistochemical staining, immunoprecipitation, complement fixation assay, flow cytometry (FACS), a protein chip method, or the like, but is not limited thereto. Preferably, an ELISA method may be used.

The SREBP2 mRNA level may be measured by amplifying SREBP2 mRNA or cDNA from a sample of a subject using a primer set or probe that specifically binds to SREBP2 mRNA, or by using hybridization with a probe to measure the presence or expression level of SREBP2 mRNA in a subject sample. The primer and the probe of SREBP2 are as described in the diagnostic composition of the present invention. The measuring of the expression level of SREBP2 mRNA may be performed using conventional methods for confirming the expression in the art without limitation. Examples of the analysis method include reverse transcription polymerase chain reaction (RT-PCR), competitive RT-PCR, real-time RT-PCR, RNase protection assay (RPA), northern blotting, microarray chip, RNA sequencing, hybridization using nanostring, in situ hybridization of tissue sections, and the like, but are not limited thereto.

In a preferred aspect of the present invention, the measuring of the expression level of SREBP2 in step (a) may be characterized by detecting the C-terminal peptide of the SREBP2 protein. The C-terminal peptide of the SREBP2 protein may refer to those described above.

(b) comparing the expression level of the SREBP2 protein or mRNA with that of a normal subject and determining an infectious disease when the expression level of the SREBP2 protein or mRNA is increased compared to the normal subject;

The expression level of SREBP2 of the subject measured by the method of step (a) described above is compared with the SREBP2 level of a normal subject measured by the same method. A subject in which the expression level of SREBP2 is increased compared to a healthy normal subject is determined to suffer from an infectious disease.

In addition, in one aspect of the present invention, it may be determined that the severity of the disease is higher as the expression level of SREBP2 is higher. With respect to the degree of increase in SREBP2 expression level, which is the reference for diagnosis, the reference of appropriate diagnosis may also be provided to exhibit the severity of the infectious disease depending on the range of the SREBP2 expression level by analyzing a correlation between the expression level of SREBP2 and the severity of the infectious disease according to a technique known in the art according to the selected method for measuring the SREBP2 expression level.

The present invention provides a use of an agent for measuring the expression level of sterol regulatory element-binding protein 2 (SREBP2) or mRNA to prepare an agent for diagnosing an infectious disease.

The present invention provides a method for diagnosing an infectious disease comprising: (a) measuring the expression level of sterol regulatory element binding protein 2 (SREBP2) or mRNA in a biological sample obtained from a subject suspected of having an infectious disease;

(b) measuring the expression level of the SREBP2 protein or mRNA in step (a);

(c) comparing the expression level of the SREBP2 protein or mRNA with that of a normal subject; and

(d) diagnosing an infectious disease when the expression level of the SREBP2 protein or mRNA is increased compared to the normal subject in step (c).

In one embodiment, the present invention provides a method for diagnosing and treating an infectious disease of a subject comprising the following steps of:

(i) measuring the expression level of sterol regulatory element binding protein 2 (SREBP2) or mRNA in a biological sample obtained from a subject suspected of having an infectious disease;

(b) measuring the expression level of the SREBP2 protein or mRNA in step (i);

(iii) comparing the expression level of the SREBP2 protein or mRNA with that of a normal subject;

(iv) diagnosing an infectious disease when the expression level of the SREBP2 protein or mRNA is increased compared to the normal subject in step (iii); and

v) treating the infectious disease by administering a therapeutic drug for treating the infectious disease to the diagnosed subject or through surgery.

Methods including steps i) to v) are understood based on the method including steps a) to d) described above.

Step v) is a step of performing the treatment of the diseases by a means, such as administration of a therapeutic drug such as ciprofloxacin and ceftriaxone, surgery, or the like, to the subject diagnosed with the disease in step iv).

The ‘treatment’ of the present invention comprehensively refers to improving hearing loss or symptoms caused by the hearing loss, and may include treating or substantially preventing the disease, or improving the conditions thereof and includes palliating, treating or preventing a symptom or most of symptoms derived from the disease, but is not limited thereto.

The type of ‘therapeutic drug’ is not particularly limited as long as the therapeutic drug is any type of drug typically used for the treatment of neurodegenerative diseases or inflammatory diseases. In addition, the therapeutic drug is administered to a subject in a ‘therapeutically effective dose’, and the therapeutically effective dose for patients may be determined by those skilled in the art by considering various factors, such as the age, weight, health condition, and sex of a patient, the severity of a disease, a diet and an excretion rate as well as unique properties, a route of administration, and a treatment number of the drug. The route of administration of the therapeutic drug is not particularly limited, and the therapeutic drug may be administered orally or parenterally, and the route of administration includes both local administration and systemic administration. The parenteral administration is not limited thereto, but may be, for example, intranasal drug application, subcutaneous injection, and the like, and as another example, a method such as intramuscular injection, intravenous injection, or the like may be used.

The ‘sample’ of the present invention is isolated and obtained from a subject suspected of having a disease, but is not limited thereto, but may be selected from the group consisting of cells, tissues, blood, serum, plasma, saliva, sputum, mucosal fluid, and urine. The ‘subject’ may be animals, preferably animals including mammals, particularly humans, and may be cells, tissues, organs, etc. derived from animals. The subject may be a patient requiring the therapeutic effect.

The term “comprising” used herein is used in the same meaning as “including” or “characterized by”, and does not exclude additional ingredients or steps of the method, which are not specifically mentioned in the composition or the method according to the present invention. In addition, the term “consisting of” means excluding additional elements, steps or ingredients, etc., unless otherwise described. The term “essentially consisting of” means including materials or steps which do not substantially affect basic properties thereof in addition to the described materials or steps within the range of the composition or the method.

### Advantageous Effects

According to the present invention, since the expression level of SREBP2 or the C-terminal peptide thereof increases in proportion to the severity of the disease in infectious diseases, it can be very useful for diagnosing these infectious diseases and predicting the severity of these infectious diseases.

### DESCRIPTION OF DRAWINGS

FIGS. 1A to 1H illustrate results of analyzing the usefulness of SREBP2 as a marker for diagnosing the severity of SARS-CoV-2 infection (COVID-19) in the blood of patients.

FIGS. 2A to 2E illustrate results of measuring relative mRNA levels of SREBF2 (FIG. 2A), SESN1 (FIG. 2B), PCSK9 (FIG. 2C), HMGCR (FIG. 2D), and LDLR (FIG. 2E).

FIGS. 3A to 3F are results of confirming that a SREBP2 C-terminal reflects the severity of an infectious disease and may be utilized as a diagnostic marker.

FIGS. 4A to 4G are results of confirming that the activation of SREBP2 is required for vascular inflammatory responses through cholesterol release and cytokine expression.

### MODES FOR THE INVENTION

Hereinafter, the present invention will be described in detail by the following Examples. However, the following Examples are just illustrative of the present invention, and the contents of the present invention are not limited to the following Examples.

Experiment Method

1. Plasma Sample

The whole blood was collected from patients admitted to Yeungnam University Hospital who were diagnosed with SARS-CoV-2 infection (COVID-19) at the Public Health Center in Daegu. Patients with COVID-19 sepsis were defined using a criteria provided by the Sepsis Consensus Conference Committee. The whole blood of patients with pneumonia and septic shock was collected from patients admitted to Yeungnam University Hospital. Healthy volunteers were used as a control. Clinical data were collected for all patients. Plasma samples were prepared by centrifugation at 2000×g for 5 minutes within 12 hours after the whole blood collection. A human study protocol was approved by the Daegu Yeungnam University Hospital Institutional Review Board.

2. Total Cholesterol, HDL-Cholesterol, and LDL-Cholesterol in Patient's Blood

Total cholesterol, HDL-cholesterol, and LDL-cholesterol levels in the patient's blood were analyzed using a modular DPE system.

3. PBMC Isolation and Incubation

Samples from healthy volunteers, patients with SARS-CoV-2 pneumonia or discharged patients were obtained from the Yeungnam University Medical Center. Heparin-treated blood samples were used in a fresh state within 4 hours, and peripheral blood mononuclear cells (PBMCs) were isolated from the blood using Ficoll-Hypaquek or NycoPrepk according to the manufacturer's recommendations. Then, the purified PBMCs were obtained using an MACSprep™ PBMC isolation kit, and incubated in RPMI-1640 containing 1 mM of sodium pyruvate, 2 mM of L-glutamine, 4.5 mg/L of glucose, 10 mM of HEPES, and 2 mg/L of sodium bicarbonate.

4. SREBP2 Transcriptional Activity Assay

The transcriptional activity of SREBP2 was determined by an ELISA method using a kit from Abcam (ab133111, Abcam) according to a manufacturer's protocol. Briefly, a nuclear extract corresponding to 30 μg of a protein content was added to each well of a 96-well plate coated with a double-stranded DNA sequence having a consensus SREBP-binding sequence (sterol regulatory element (SRE)). The nuclear extract was hybridized with the coated double-stranded DNA sequences with the consensus SRE on the plate overnight at 4° C. An activated SREBP transcription factor complex was detected at 450 nm after addition of a primary antibody specific for SREBP2 and a secondary antibody conjugated to HRP.

5. NF-kB Transcriptional Activity Assay

As known in the related art, the preparation of the nuclear extract and TransAM analysis were performed. The activity of individual NF-κB subunits was determined using an ELISA-based NF-κB family transcription factor assay kit. Briefly, the nuclear extract (2 μg) was added and incubated in a 96-well plate coated with NF-κB consensus oligonucleotides. The captured complex was incubated with a specific NF-κB primary Ab and then detected using an HRP-conjugated secondary antibody included in the kit. Finally, OD values were measured at 450 nm.

6. SREBP2 C-Terminal ELISA

A competitive ELISA was performed using an antibody recognizing an SREBP2 C-terminal. An SREBP2 C-terminal (639-1031aa) protein was diluted to 2 μg/100 μl, coated on a Nunc-Immuno™ MicroWell™ 96 well plate, and incubated overnight at 4° C. Before using, the plate was washed 3 times with PBST and blocked with 3% BSA at 37° C. for 30 minutes. The primary antibody (1:2000 dilution) and plasma samples (20 μg) were pre-incubated at 37° C. for 1 hour, then the pre-incubated samples were transferred to a peptide-coated plate and incubated at 37° C. for 1 hour. The plate was washed 5 times with PBST. The secondary antibody (1:5000 dilution) was incubated at 37° C. for 30 minutes, and then the plate was washed 5 times with PBST. The washed plate was treated with 100 μl/well of a TMB ELISA substrate at 37° C. for 10 minutes, and then 100 μl/well of a stop solution was added. The detection was performed at 450 nm with a microplate reader.

Experimental Results

1. SREBP2 was Highly Activated in PBMCs of Patients with SARS-CoV-2 Infection, and Subsequently had a Cytotoxic Effect on PBMCs

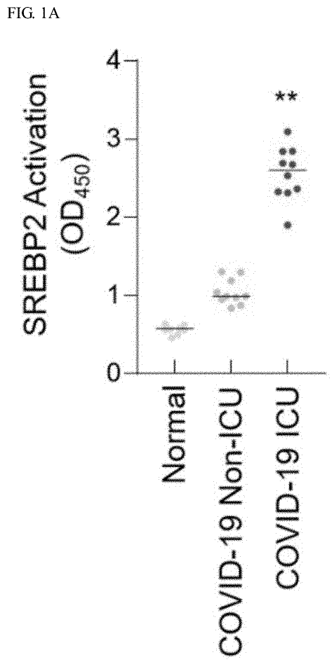

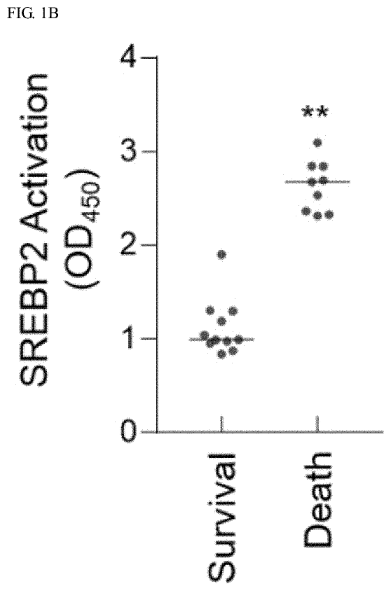

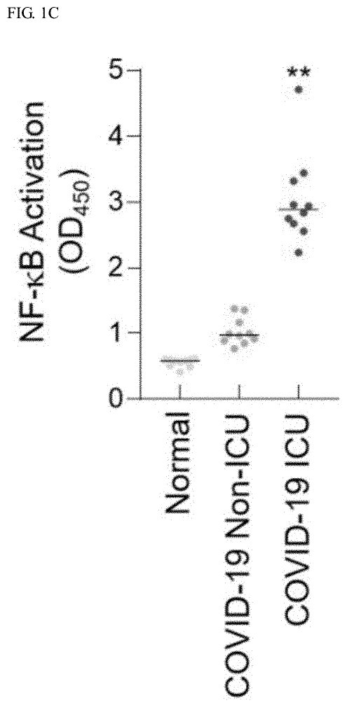

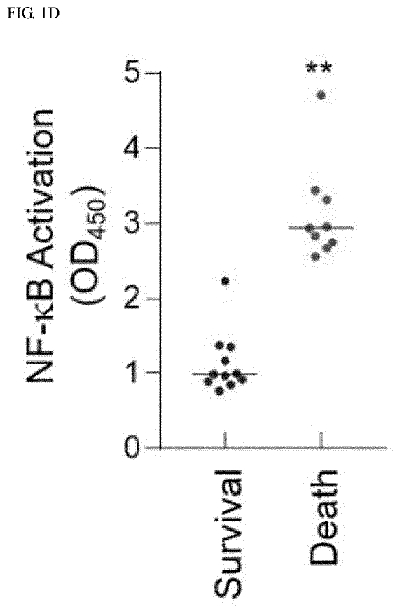

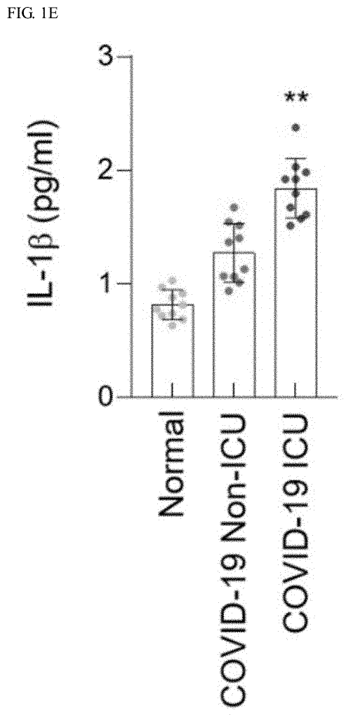

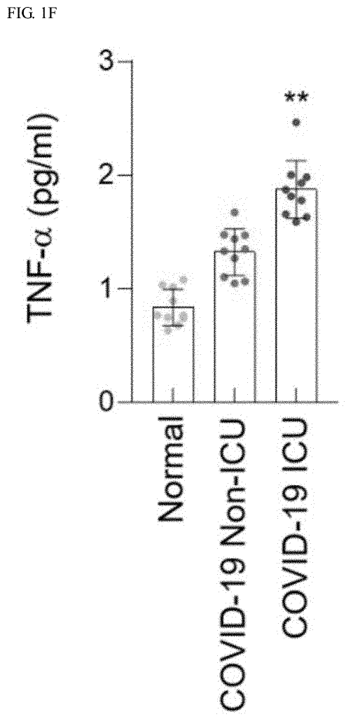

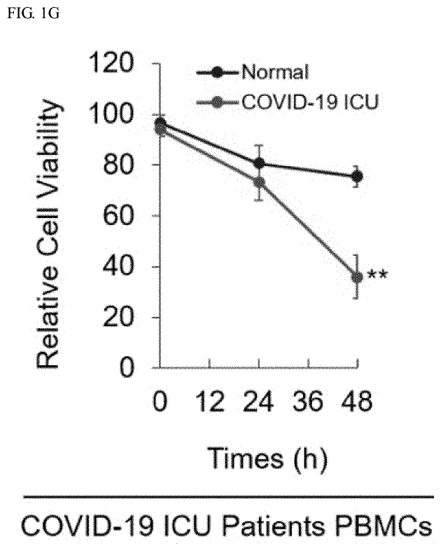

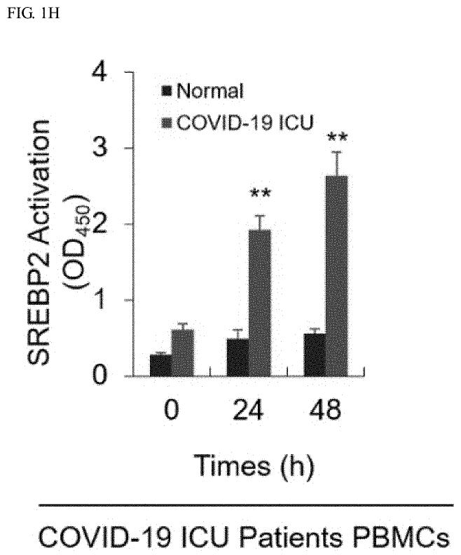

In the blood of patients with SARS-CoV-2 infection (COVID-19), the levels of total cholesterol (Ch), high-density lipoprotein (HDL-Ch), and low-density lipoprotein (LDL-Ch) were lower than those of a normal subject, and were lower in intensive care unit (ICU) patients than in non-ICU patients (results not shown). There were no significant associated diseases in each group. According to the analysis, the SREBP2 activity increased as the severity of SARS-CoV-2 infection increased from non-ICU to ICU (FIG. 1A), which was inversely proportional to the tendency in cholesterol levels. The activation level of SREBP2 was increased in dead patients than in living patients (FIG. 1B), and accordingly, SREBP2 may be suggested as an indicator of the severity of SARS-CoV-2 infection.

In addition, NF-κB, known as a crosstalk molecule of SREBP2, showed a similar increasing tendency as the severity of SARS-CoV-2 infection increased (FIGS. 1C and 1D). As the severity of SARS-CoV-2 infection increased, the production of inflammatory cytokines such as IL-1β and TNF-α by SREBP2 or NF-κB also increased (FIGS. 1E and 1F).

When PBMCs from patients infected with SARS-CoV-2 were incubated in vitro, the viability of PBMCs isolated from the blood of ICU patients with SARS-CoV-2 infection and the blood of patients with acute respiratory syndrome (ARDS) decreased more rapidly over time than that of a normal subject (FIG. 1G). In addition, the activation level of SREBP2 increased in incubated PBMCs over time (FIG. 1H).

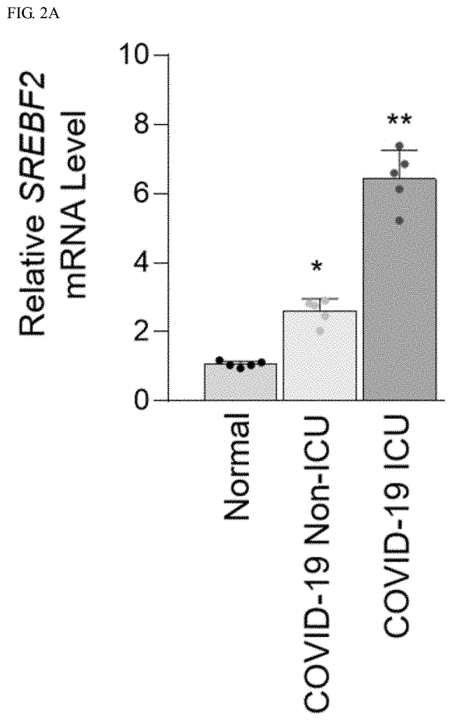

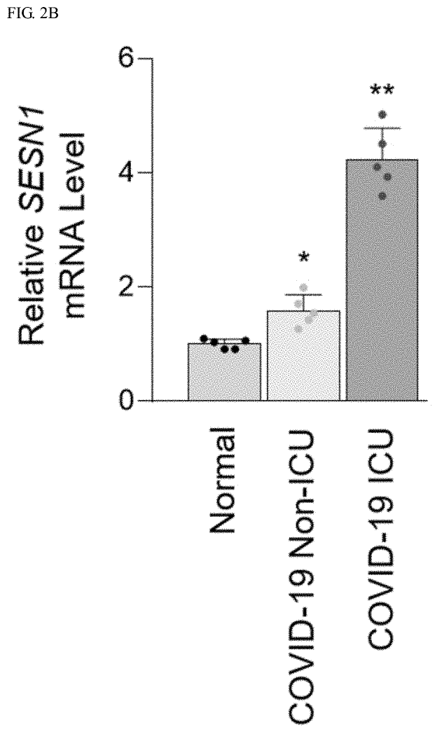

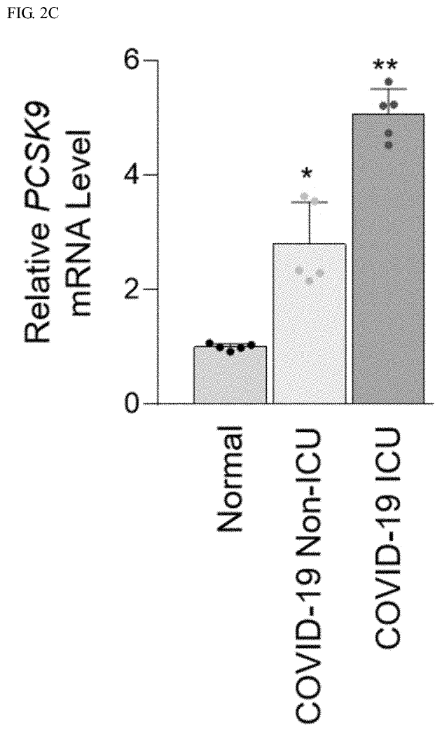

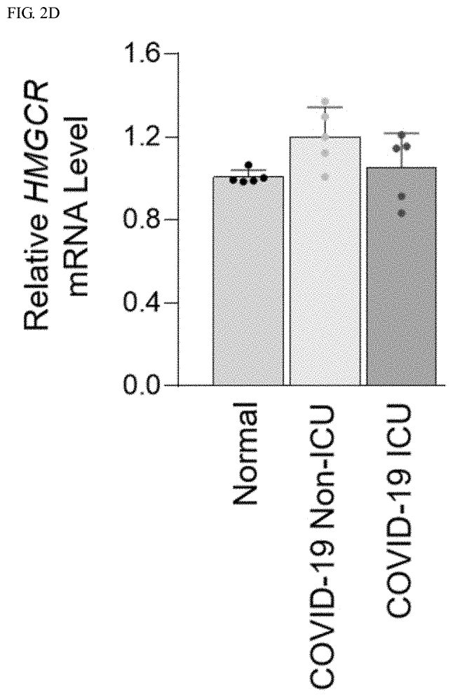

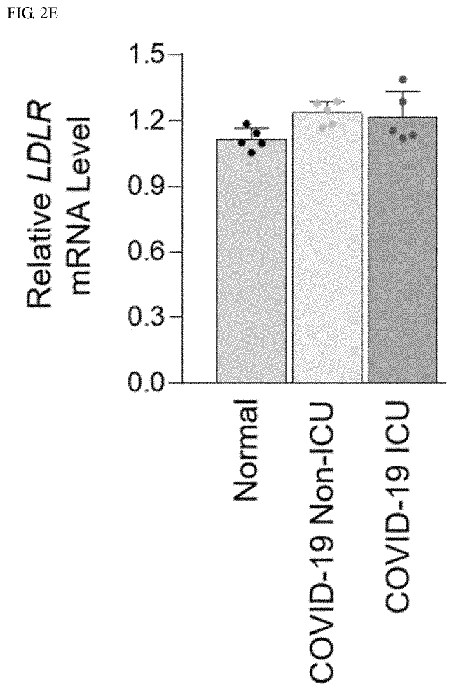

According to the qRT-PCR result, SREBF2 mRNA was increased in a severity-dependent manner in patients with SARS-CoV-2 infection, and the levels of sestrin 1 (SESN1) and proprotein convertase subtilisin/kexin type 9 (PCSK9), which were known to regulate lipids, also showed a similar increasing trend as the severity of SARS-CoV-2 infection increased (FIG. 2). On the other hand, the mRNA level of 3-hydroxy-3-methylglutaryl-CoA reductase (HMGCR), an enzyme that acts upstream of cholesterol synthesis, and a low-density lipoprotein receptor (LDLR) did not change regardless of the severity of SARS-CoV-2 infection (FIG. 2). These results suggest that SARS-CoV-2 infection increases the activity of SREBP2 as an inflammatory transcription factor, while a direct synthesis pathway of cholesterol by SREBP2 is inhibited.

2. Expression Level of SREBP2 C-Terminal Reflects Severity of SARS-CoV-2 Infection.

The role of the SREBP2 N-terminal has been confirmed in many studies. When the SREBP2 N-terminal and C-terminal are cleaved by S113 and S2P, the N-terminal move to the nucleus to ultimately regulate the synthesis of cholesterol. However, the role of the SREBP2 C-terminal has not yet been reported.

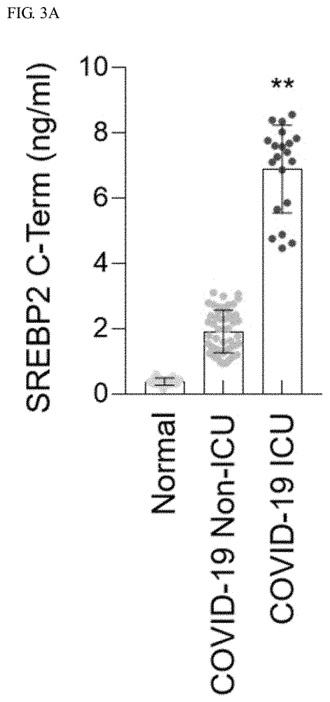

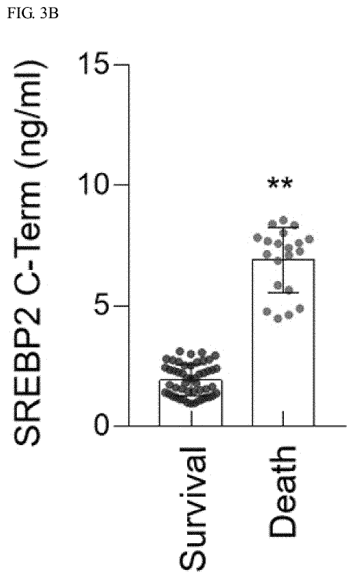

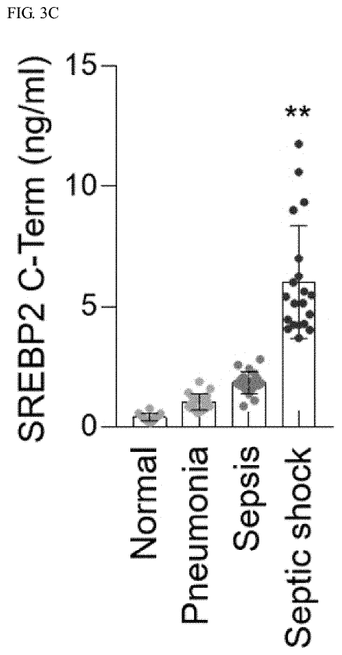

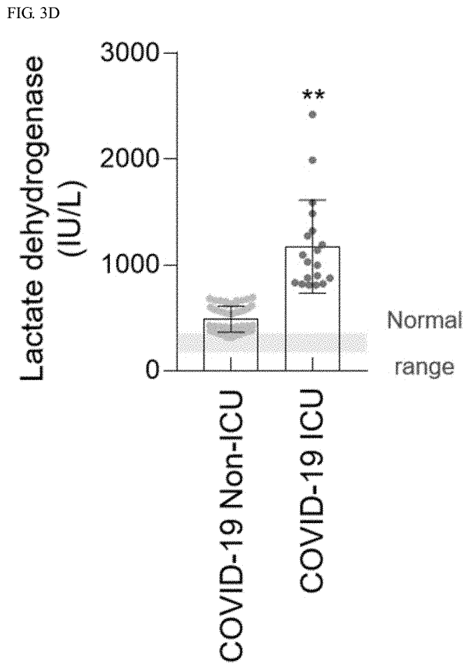

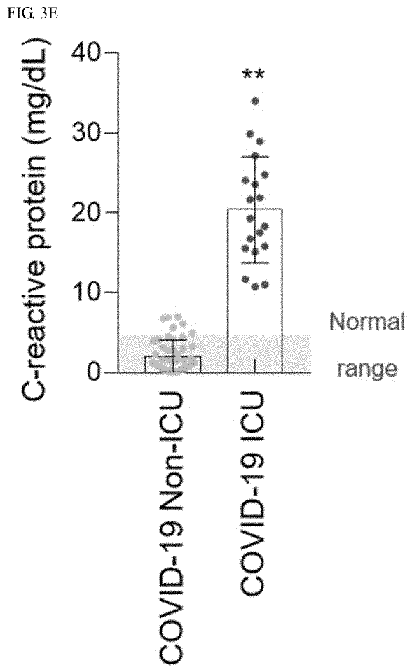

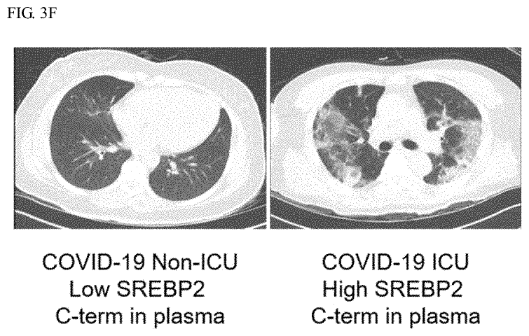

The present inventors hypothesized that the SREBP2 C-terminal should be released from the blood of SARS-CoV-2 infected patients in response to the activation degree of SREBP2. In particular, the level of the SREBP2 C-terminal increased rapidly in patients with severe SARS-CoV-2 infection, including ICU patients and dead patients (FIGS. 3A and 3B). The SREBP2 C-terminal was also released in severe sepsis (septic shock) (FIG. 3C), which suggested the usability of the SREBP2 C-terminal as a general marker for infectious diseases.

This usability is supported by increased levels of lactate dehydrogenase (LDH) and C-reactive protein (CRP) in the blood of SARS-CoV-2 infected patients (FIGS. 3D and 3E). Such a high level of the SREBP2 C-terminal was closely associated with hyperinflammation in lung tissues of patients with SARS-CoV-2 infection. Computed tomography (CT) images of ICU patients with high SREBP2 C-terminal levels in the plasma (right panel of FIG. 3G) had more severe lung inflammation than non-ICU patients with low SREBP2 C-terminal levels (left panel of FIG. 3F).

3. SREBP2 C-Terminal is an Indicator of an Uncontrolled Vasculature and May be Protected by Pharmacological Inhibition or Knockdown of SREBP2.

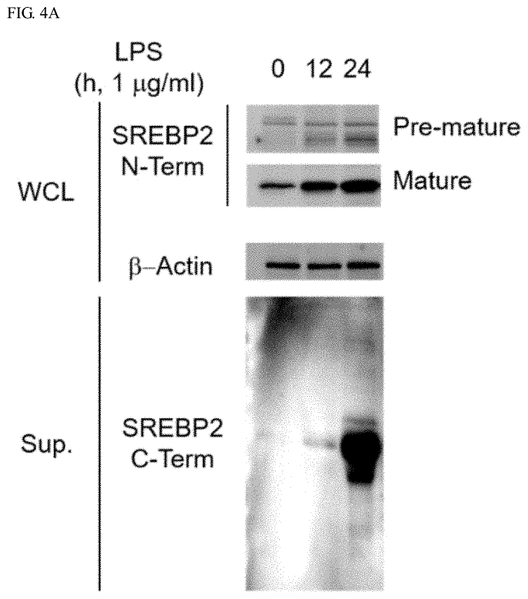

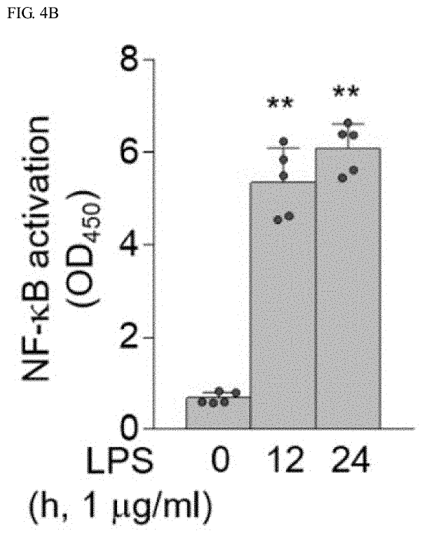

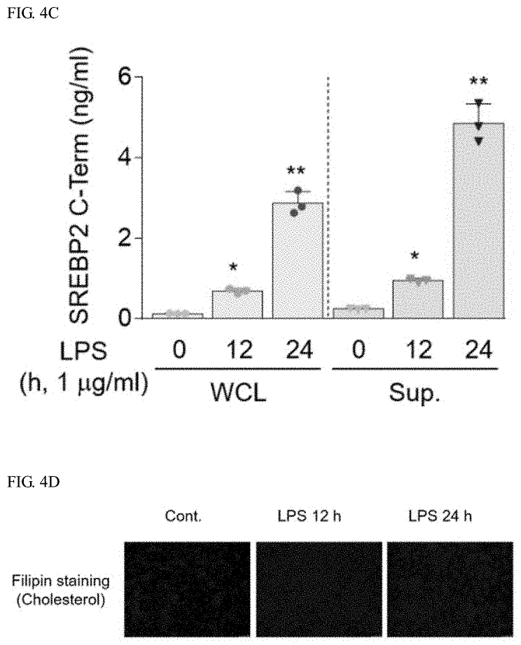

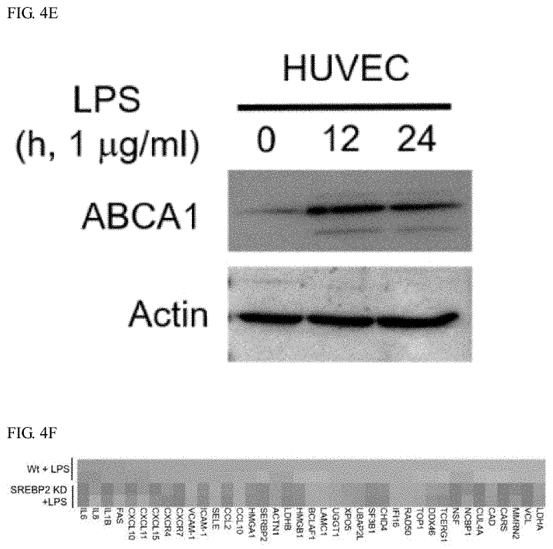

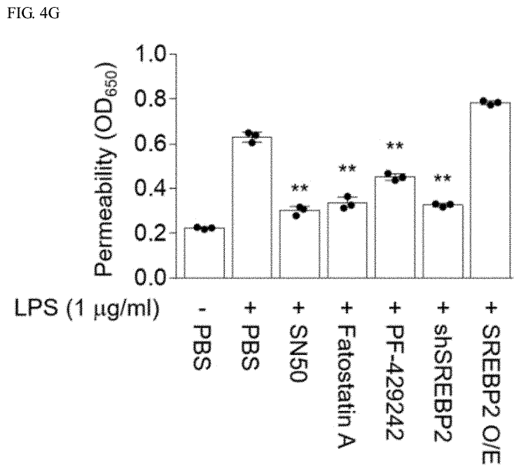

Western blot assay was performed to demonstrate different translocation targets of the N-terminal and C-terminal of SREBP2. Upon exposure to LPS, the expression of the N-terminal of SREBP2 transiently increased with time in a whole cell lysate (WCL) of HUVECs (FIG. 4A). On the other hand, the SREBP2 C-terminal was highly expressed in a supernatant at the end of LPS stimulation (24 hr) (FIG. 4A). Similarly, in other cell types such as HEK293 and HUVEC, the SREBP2 N-terminal was detected in the cell lysate, but was not observed in a culture medium (results not shown). Unlike SREBP2 as a late mediator, the NF-κB activation by LPS was increased at an early age. This is interpreted as mediating severe lung damage by a crosstalk between NF-κB and SREBP2. The SREBP2 C-terminal was detectable in both the WCL and the culture supernatant, but the level was higher in the supernatant (FIG. 4A).

A notable difference between the N-terminal and the C-terminal of SREBP2 was an increasing ratio with simulation time. This was consistent with time-dependent NF-κB activation upon LPS treatment (FIG. 4B). In contrast to the monotonic increase in SREBP2 N-terminal, the SREBP2 C-terminal increased dramatically at 24 hr after LPS stimulation (FIG. 4C).

The duration of LPS stimulation induced different results in cholesterol metabolism. Through Filipin staining for visualizing intracellular cholesterol, it was confirmed that cholesterol was accumulated in HUVECs after 12 hours of LPS stimulation (FIG. 4D). However, the cholesterol level was decreased after 24 hr of LPS stimulation (FIG. 4D). As a result of western blot assay of an ATP-binding cassette transporter (ABCA1), also known as a cholesterol efflux regulatory protein (CERP), a more reduced expression was confirmed after 24 hours than after 12 hours (FIG. 4E).

In HUVECs, LPS stimulation induces upregulated release of inflammatory cytokines (top panel of FIG. 4F). However, genetic knockout of SREBP2 was able to suppress the cytokine storm even after LPS stimulation (lower panel of FIG. 4F). Pharmacological inhibition of NF-κB, SREBP2 and S113 (PF-429242) and shRNA of SREBP2 inhibited vascular bather destruction even under LPS stimulation (FIG. 4G). SREBP2-overexpressing (SREBP2 O/E) HUVECs were more severely damaged by LPS (FIG. 4G).

### INDUSTRIAL APPLICABILITY

According to the present invention, since the expression level of SREBP2 or the C-terminal peptide thereof increases in proportion to the severity of the disease in infectious diseases, these markers can be very useful for diagnosing these infectious diseases and predicting the severity of these infectious diseases and thus, the industrial applicability is very high.

1. A composition for diagnosing an infectious disease comprising an agent for measuring the expression level of sterol regulatory element-binding protein 2 (SREBP2) or mRNA.

2. The composition of claim 1, wherein the SREBP2 protein consists of an amino acid sequence of SEQ ID NO: 1.

3. The composition of claim 1, wherein the SREBP2 protein is a C-terminal peptide of SREBP2.

4. The composition of claim 3, wherein the C-terminal peptide of SREBP2 consists of an amino acid sequence of SEQ ID NO: 3.

5. The composition of claim 1, wherein the infectious disease is caused by one or more infections selected from the group consisting of viruses, bacteria and fungi.

6. The composition of claim 1, wherein the infectious disease is at least one selected from the group consisting of sepsis, septic shock, severe acute respiratory syndrome coronavirus 2 (SARS-CoV-2) infection, severe acute respiratory syndrome coronavirus (SARS-CoV) infection, Middle East respiratory syndrome (MERS), salmonellosis, food poisoning, typhoid, paratyphoid, systemic inflammatory response syndrome (SIRS), multiple organ dysfunction syndrome (MODS), pneumonia, pulmonary tuberculosis, tuberculosis, cold, influenza, airway infection, rhinitis, nasopharyngitis, otitis media, bronchitis, lymphadenitis, parotitis, lymphadenitis, cheilitis, stomatitis, arthritis, myositis, dermatitis, vasculitis, gingivitis, periodontitis, keratitis, conjunctivitis, wound infection, peritonitis, hepatitis, osteomyelitis, cellulitis, meningitis, encephalitis, brain abscess, encephalomyelitis, meningitis, osteomyelitis, nephritis, carditis, endocarditis, enteritis, gastritis, esophagitis, duodenitis, colitis, urinary tractitis, cystitis, vaginitis, cervicitis, salpingitis, erythema infectious, dysentery, abscesses and ulcers, bacteremia, diarrhea, dysentery, enterogastritis, gastroenteritis, genitourinary abscess, open wound or injury infection, suppurative inflammation, abscess, boil, pyoderma, impetigo, folliculitis, cellulitis, postoperative wound infection, skin laceration syndrome, skin burn syndrome, thrombotic thrombocytopenia, hemolytic uremic syndrome, renal failure, pyelonephritis, glomerulonephritis, nervous system abscess, otitis media, sinusitis, pharyngitis, tonsillitis, mastoiditis, cellulitis, devotion infection, dacryocystitis, pleurisy, abdominal abscess, liver abscess, cholecystitis, spleen abscess, pericarditis, myocarditis, placenta, amniotic fluid infection, mammitis, mastitis, puerperal fever, toxic shock syndrome, lyme disease, gas gangrene, atherosclerosis, Mycobacterium avium syndrome (MAC), enterohemorrhagic Escherichia coli (EHEC) infection, enteropathogenic Escherichia coli (EPEC) infection, enterohemorrhagic Escherichia coli infection (EIEC), methicillin-resistant Staphylococcus aureus (MRSA) infection, vancomycin-resistant Staphylococcus aureus (VRSA) infection, and listerosis.

7. The composition of claim 1, wherein the composition is a composition for diagnosing the severity of the infectious disease.

8. A kit for diagnosing an infectious disease comprising the agent for measuring the expression level of sterol regulatory element-binding protein 2 (SREBP2) or mRNA according to claim 1.

9. A method for detecting SREBP2 to provide information necessary for the diagnosis of an infectious disease, comprising:
(a) measuring the expression level of sterol regulatory element binding protein 2 (SREBP2) or mRNA in a biological sample provided from a patient suspected of having an infectious disease; and
(b) comparing the expression level of the SREBP2 protein or mRNA with that of a normal subject and determining an infectious disease when the expression level of the SREBP2 protein or mRNA is increased compared to the normal subject.

10. The method of claim 9, wherein the biological sample is at least one selected from the group consisting of blood, plasma, serum, saliva, nasal fluid, sputum, synovial fluid, amniotic fluid, ascites, cervical or vaginal secretions, urine and cerebrospinal fluid.

11. The method of claim 9, wherein in step (b), it is determined that the higher the expression level of SREBP2, the higher the severity of the infectious disease.

12. A composition for diagnosing an infectious disease comprising the agent for measuring the expression level of sterol regulatory element-binding protein 2 (SREBP2) or mRNA according to claim 1.

13. (canceled)

14. (canceled)

15. A method for diagnosing an infectious disease comprising the steps of:
(a) measuring the expression level of sterol regulatory element binding protein 2 (SREBP2) or mRNA in a biological sample obtained from a subject suspected of having an infectious disease;
(b) measuring the expression level of the SREBP2 protein or mRNA in step (a);
(c) comparing the expression level of the SREBP2 protein or mRNA with that of a normal subject; and
(d) diagnosing an infectious disease when the expression level of the SREBP2 protein or mRNA is increased compared to the normal subject in step (c).

16. The method of claim 15, wherein the SREBP2 protein consists of an amino acid sequence of SEQ ID NO: 1.
